(19) United States
(12) Patent Application Publication
(10) Pub. No.: US 2022/0076836 A1
(43) Pub. Date: Mar. 10, 2022

(54) Pan-Indication Gradient Boosting Model Using Tumor Kinetics for Survival Prediction

(71) Applicants: Genentech, Inc., South San Francisco, CA (US); Institut Roche SAS, Boulogne-Billancourt (FR)

(72) Inventors: James Lu, San Francisco, CA (US); Jin Yan Lin, Foster City, CA (US); Hui Min Phillis Chan, San Francisco, CA (US); Rene Bruno, Marseille (FR)

(21) Appl. No.: 17/469,835

(22) Filed: Sep. 8, 2021

Related U.S. Application Data
(60) Provisional application No. 63/104,313, filed on Oct. 22, 2020.
(60) Provisional application No. 63/075,710, filed on Sep. 8, 2020.

(51) Int. Cl. G16H 50/20; G16H 50/50; A61B 5/00

(52) U.S. Cl. CPC G16H 50/20; G16H 50/50; A61B 5/4848; A61B 5/7275; A61B 5/4842

(57) ABSTRACT

A method for predicting a clinical outcome for a subject. Input data for a group of features is formed using baseline data and tumor kinetic data derived from longitudinal data for a set of variables for the subject, the set of variables including tumor size. A clinical outcome output that provides an indication of the clinical outcome for the subject is generated using a pan-indication model and the input data. The pan-indication model includes a gradient boosting decision tree-based ensemble machine learning algorithm.

### CROSS-REFERENCE TO RELATED APPLICATION

This application is related to U.S. Provisional Patent Application No. 63/104,313, filed Oct. 22, 2020, and to U.S. Provisional Patent Application No. 63/075,710, filed Sep. 8, 2020, both of which are incorporated herein by reference in their entireties.

### FIELD

This description is generally directed towards predicting a clinical outcome for a subject based on tumor size. More specifically, methods and systems are disclosed for predicting clinical outcomes for subjects having tumors using a pan-indication model and tumor kinetic data derived from longitudinal tumor size data, the pan-indication model including a gradient boosting machine learning algorithm.

### BACKGROUND

Tumors develop when cells in the body reproduce too quickly. Tumors can be benign (not cancerous), premalignant (not yet cancerous), or malignant (cancerous). Different types of tumors can affect patients in different ways, which may lead to differences in patient survival. Overall survival may be one manner of characterizing the impact of a tumor(s) on a patient. The overall survival associated with a cancer may be, for example, the length of time from either the date of diagnosis or the start of treatment for the cancer that patients diagnosed with the tumor are still alive.

Some currently available methodologies use machine learning models to predict survival (e.g., overall survival) in patients with tumors. However, some of these machine learning prediction models may not provide desired, clinically reliable levels of predictive performance. In addition, machine learning prediction models are currently generated as cancer-specific. For example, a machine learning prediction model for predicting patient survival provides insights for a singular type of cancer. Accordingly, an independent machine learning model is used for each type of cancer. For instance, a machine learning model for predicting lung cancer survival is separate and may not be interchangeable with a machine learning model for predicting breast cancer survival. And such a machine learning model for predicting lung cancer survival may be unable to reliably predict breast cancer survival.

By extension, because treatments between cancers typically vary, machine learning prediction models for predicting response to treatment are also particular to the type of cancer, the type of treatment, and/or the treatment protocol. Thus, currently available methods for developing machine learning prediction models can entail designing and creating, for each type of cancer and that cancer's corresponding treatment protocol, a different machine learning prediction model. This type of development can be expensive, time-consuming, and inefficient. Further, this type of approach may mean that machine learning prediction models are unavailable for certain types of cancers or certain treatment protocols. Thus, it may be desirable to have methods and systems that recognize and consider these issues.

### SUMMARY

In various embodiments, a method is provided for predicting a clinical outcome for a subject. Longitudinal data is identified for a set of variables for the subject, the set of variables including tumor size. A trajectory of the set of variables is modeled over a period of time using the longitudinal data to derive values for a plurality of tumor kinetic parameters. Input data is formed for a group of features using the values for the plurality of tumor kinetic parameters and baseline data. The clinical outcome for the subject is predicted using a pan-indication gradient boosting model and the input data.

In various embodiments, a method for predicting a clinical outcome for a subject is provided. Input data for a group of features is formed using baseline data and tumor kinetic data derived from longitudinal data for a set of variables for the subject, the set of variables including tumor size. A clinical outcome output that provides an indication of the clinical outcome for the subject is generated using a pan-indication model and the input data. The pan-indication model includes a gradient boosting decision tree-based ensemble machine learning algorithm.

In various embodiments, a method for generating a clinical outcome output for a subject having a tumor is provided. Input data for a group of features is formed using baseline data and tumor kinetic data derived from longitudinal data for a set of variables for the subject, the set of variables including tumor size. The group of features includes at least three features selected from features comprising (listed in order of decreasing impact on the clinical outcome):

tumor growth rate (KG),

C-reactive protein level (CRP),

time to tumor regrowth (TTG),

baseline neutrophil/lymphocyte ratio (BNLR),

baseline Eastern Cooperative Oncology Group score (BECOG),

liver metastasis level (LIVER),

tumor shrinkage rate (KS),

hemoglobin level (HGB),

time since initial diagnosis (TSD),

total protein level (TPRO),

albumin (ALBU), and

number of metastatic sites at enrollment (METSITES).

The clinical outcome output for the subject is generated using a pan-indication model and the input data. The pan-indication model includes a gradient boosting decision tree-based ensemble machine learning algorithm.

In various embodiments, a method for predicting clinical outcomes for subjects having tumors is provided. Training data is identified for a plurality of initial features for a plurality of training subjects. The plurality of initial features includes a plurality of tumor kinetic parameters and a plurality of baseline parameters. At least a portion of the training data is transformed to form input data for a plurality of training features. A pan-indication gradient boosting model is trained, via the input data, to generate a clinical outcome output using a feature subset of the plurality of training features. The clinical outcome output for a subject having a tumor is generated using the pan-indication gradient boosting model that has been trained and input data for the feature subset.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a clinical outcome prediction system in accordance with one or more example embodiments.

FIG. 2 is a flowchart of a process for predicting a clinical outcome for a subject in accordance with various embodiments.

FIG. 3 is a flowchart of a process for forming input data for a pan-indication model in accordance with various embodiments.

FIG. 4 is a flowchart of a process for training a pan-indication model and using the trained pan-indication model to predict clinical outcomes in accordance with various embodiments.

FIG. 5 is a block diagram of a computer system in accordance with various embodiments.

FIG. 6 is a plot illustrating the relative importance of features used as input for a pan-indication model in accordance with various embodiments.

FIG. 7 is a plot illustrating the accuracy of the pan-indication model as compared to other models in accordance with various embodiments.

FIG. 8 is a plot illustrating an ability of XGBoost to stratify patients based on individual predictions in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Current machine learning models predict outcomes for a single type of tumor (e.g., type of cancer) or a single treatment protocol (e.g., which may include a treatment type). But there are many different types of cancers and many different types of tumors, as well as many different types of treatments, each of which may be associated with one or more different types of treatment protocols. Using the current approach for machine learning models may therefore translate into a time-consuming process of developing prediction models for each type of tumor (e.g., cancer) or treatment protocol (or treatment type). In addition, the need to develop prediction models that are specific to a type of tumor or treatment protocol means that such prediction models that can reliably predict outcomes with a desired level of clinical accuracy may be restricted to those tumor types and/or treatment protocols for which sufficient data is present. Thus, such prediction models may be unable to be constructed for certain tumor (e.g., cancer) types, treatment protocols (or treatment types), or both that are novel or newly identified and/or where observation data is limited or unavailable.

Further, some subjects (or patients) may suffer from more than one cancer (e.g., suffer from multiple tumors of different types), undergo more than one type of treatment, or both at generally a same time. Cancer-specific or treatment-specific prediction models may be unable to account for the compound impact of multiple cancers and/or treatments when predicting clinical outcomes for these types of subjects.

Thus, it may be desirable to have a methodology and/or system for accurately predicting a clinical outcome for a subject having a tumor independent of the type of tumor or the treatment protocol selected for or being administered to the subject. Accordingly, the methods and systems described herein use machine learning models that enable simplified, accurate predictions of clinical outcomes for subjects, independent of tumor type of treatment protocol. For example, the embodiments described herein provide pan-indication machine learning models built to provide accurate predictions of clinical outcomes for subjects, independent of the type of tumor (or cancer) and independent of the type of treatment protocol that has been or is being administered to the subjects.

The pan-indication machine learning models described herein ensure that predictions can be provided for tumors (e.g., cancer) and/or treatment protocols for which specific machine learning models have not been developed or for which little preexisting, reliable, or usable data exists. In addition, these pan-indication machine learning models may provide survival predictions for novel and/or newly identified tumors and can account for multiple cancers or treatment protocols relating to a subject. Further, the pan-indication machine learning models described herein may enable predictions of clinical outcomes over a variety of tumor types and treatment protocols with a higher level of accuracy and clinical reliability as compared to currently available prediction models that are tumor-specific and/or treatment-specific.

Recognizing and considering the importance and utility of a tumor-agnostic and treatment protocol-agnostic machine learning model that can accurately predict a clinical outcome, such as overall survival, for a subject having a tumor, various embodiments of machine learning-based systems and methods are described herein for generating such predictions using longitudinal tumor-related data. More particularly, various embodiments of methods and systems are described herein for predicting clinical outcomes via a pan-indication model that uses input data for a group of features formed using baseline data and tumor kinetic data derived from longitudinal tumor size data. The longitudinal tumor size data includes data regarding tumor size that is processed and used to form the input data for the group of features. The pan-indication model includes a gradient boosting algorithm. The gradient boosting algorithm may include, for example, a gradient boosting decision tree-based ensemble machine learning algorithm.

In one or more examples, the various embodiments described herein provide a pan-indication Extreme Gradient Boosting (XGBoost) model for predicting a clinical outcome for a subject in a treatment-independent and disease-independent manner. The prediction of clinical outcome may include, for example, an overall survival for the subject. The predicted clinical outcome may be used to, for example, without limitation, generate hazard ratio predictions, make decisions regarding personalized health care (e.g., whether or not to switch or adjust a treatment protocol), or a combination thereof. Training of the pan-indication XGBoost model can be performed using data collected from a plurality of clinical trials across multiple tumor types (or disease/cancer types). Similarly, training of the pan-indication XGBoost model can be performed using data collected from a plurality of clinical trials across multiple tumor types and multiple treatment types.

The pan-indication XGBoost model is trained to generate a clinical outcome output using features that include, for example, tumor kinetic parameters. Exemplary tumor kinetic parameters may include tumor growth inhibition (TGI) metrics. The clinical outcome output provides an indication of clinical outcome (e.g., overall survival), thus enabling a prediction of clinical outcome. Of note, the present pan-indication model may differ from prior survival prediction models in that the trained model refrains from using features that include or otherwise indicate tumor type or treatment protocol/type. In this manner, the clinical outcome output of the present pan-indication model is independent or agnostic of tumor type (or disease/cancer type) or treatment protocol/type.

Further, the various embodiments described herein provide methods and systems that improve the functioning of a computing platform (e.g., computer system, cloud computing platform, etc.) used to predict the clinical outcome for a subject. For example, using a pan-indication model that has been trained with input data formed using both baseline data and tumor kinetic data as described herein improves the accuracy of the predicted clinical outcome by the computing platform. Further, using the pan-indication model may reduce the overall amount of processing resources (e.g., memory, power, etc.) needed to generate such predictions because multiple models do not need to be trained, stored for use, etc. for the different possible types of cancer and/or treatment protocols (or types). Still further, using the pan-indication model that has been trained as described herein may enable generating predicted clinical outcomes for subjects having novel or newly identified tumors or tumors for which little to no data is available or for treatment protocols for which little to no data is available, which might be otherwise impossible for a computing platform that only uses tumor-specific or treatment-specific machine learning models.

### II. Pan-Indication Model

IIA. Clinical Outcome Prediction System

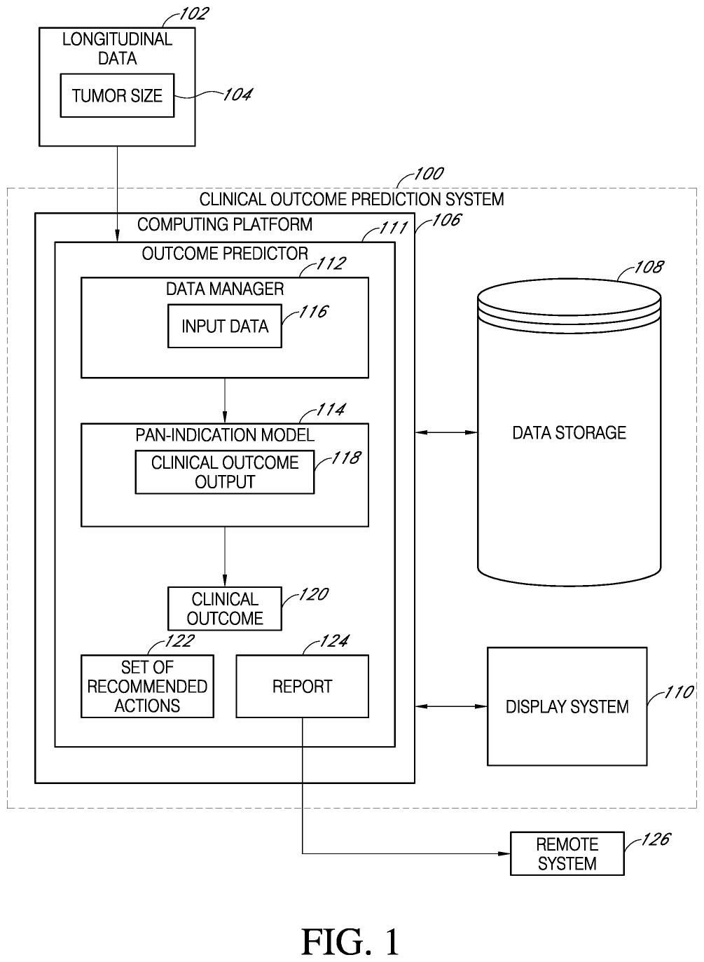

FIG. 1 is a block diagram of a clinical outcome prediction system 100 in accordance with various embodiments. Clinical outcome prediction system 100 is used to predict clinical outcomes for subjects who have tumor growth (e.g., one or more tumors). More particularly, clinical outcome prediction system 100 is used to predict one or more clinical outcomes for a subject given that subject's tumor growth or change in size over a period of time. Clinical outcome prediction system 100 may be used in a hospital setting to predict clinical outcomes for subjects receiving treatment, a clinical trial setting to predict clinical outcomes for subjects undergoing a clinical trial, or a different type of research setting.

In various embodiments, clinical outcome prediction system 100 is used to predict one or more clinical outcomes for a subject as a result of the administration of a treatment protocol based on longitudinal data 102 that is tumor-related. Longitudinal data 102 includes, for example, longitudinal data for tumor size 104. Longitudinal data 102 for tumor size 104 may provide data or an indication of a tumor's size over a given period of time.

Clinical outcome prediction system 100 includes computing platform 106, data storage 108, and display system 110. Computing platform 106 may take various forms. In one or more embodiments, computing platform 106 includes a single computer (or computer system) or multiple computers in communication with each other. In other examples, computing platform 106 takes the form of a cloud computing platform.

Data storage 108 and display system 110 are each in communication with computing platform 106. In some examples, data storage 108, display system 110, or both may be considered part of or otherwise integrated with computing platform 106. Thus, in some examples, computing platform 106, data storage 108, and display system 110 may be separate components in communication with each other, but in other examples, some combination of these components may be integrated together. Communication between the different components may be implemented using any number of wired communications links, wireless communications links, optical communications links, or a combination thereof.

Clinical outcome prediction system 100 includes outcome predictor 111. Outcome predictor 111 may be implemented using software, hardware, firmware, or a combination thereof. In one or more embodiments, outcome predictor 111 includes data manager 112 and pan-indication model 114. Pan-indication model 114 is a machine learning model that may be comprised of any number of or combination of models, algorithms, equations, formulas, etc. Pan-indication model 114 may be a tumor-agnostic and/or treatment-agnostic model for predicting clinical outcomes for subjects. In one or more embodiments, pan-indication model 114 includes a pan-indication gradient boosting model. This pan-indication gradient boosting model may include, for example, a gradient boosting decision tree-based ensemble machine learning algorithm (e.g., XGBoost).

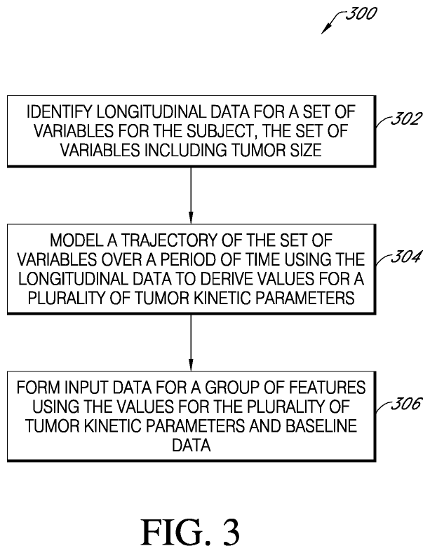

Data manager 112 obtains longitudinal data 102 and forms (e.g., creates) input data 116 to send into pan-indication model 114. Longitudinal data 102 may be received from a remote system, may be retrieved from a data store, may be retrieved from data storage 108, may be input by a user, or may be obtained in some other manner. One example of an implementation for forming input data 116 based on longitudinal data 102 is described with respect to FIG. 3 below.

During a training stage, input data 116 includes training input data that is used to train pan-indication model 114 to generate clinical outcome output 118 with a desired level of accuracy. Once pan-indication model 114 has been trained, pan-indication model 114 can be used to generate clinical outcome output 118 for a current or future subject using input data 116 specific to that subject.

Clinical outcome output 118 may take different forms that relate to a clinical outcome 120 for the subject. Clinical outcome 120 may include or take the form of overall survival. In some embodiments, clinical outcome output 118 includes a score that provides an indication of clinical outcome 120. For example, clinical outcome output 118 may include a score that indicates overall survival. In some embodiments, clinical outcome 120 may include a score that can be further processed and used to predict clinical outcome 120. For example, outcome predictor 111 may convert clinical outcome output 118 into a prediction of clinical outcome 120.

In one or more embodiments, outcome predictor 111 generates set of recommended actions 122 based on clinical outcome 120. Set of recommended actions 122 may include, for example, at least one of a recommendation to switch treatment protocols, a recommendation to adjust a current treatment protocol, a recommendation to adjust a treatment dosage, a recommendation to adjust a treatment interval, a recommendation to add a different treatment protocol to the current treatment protocol, or a recommendation to perform some other type of personalized health care action.

Outcome predictor 111 may visually present clinical outcome 120 using display system 110. In some cases, outcome predictor 111 may additionally, or alternatively, visually present at least a portion of clinical outcome output 118, set of recommended actions 122, or both using display system 110. In some embodiments, outcome predictor 111 generates report 124 that includes clinical outcome 120, clinical outcome output 118 (to the extent it is different from or does not include clinical outcome 120), set of recommended actions 122, or a combination thereof. Report 124 may be displayed on display system 110, sent to a remote system 126 (e.g., a mobile device, a laptop, a server, a computer, a cloud computing platform, etc.), or both.

IIB. Exemplary Methodologies for Predicting Clinical Outcomes Using a Pan-Indication Model

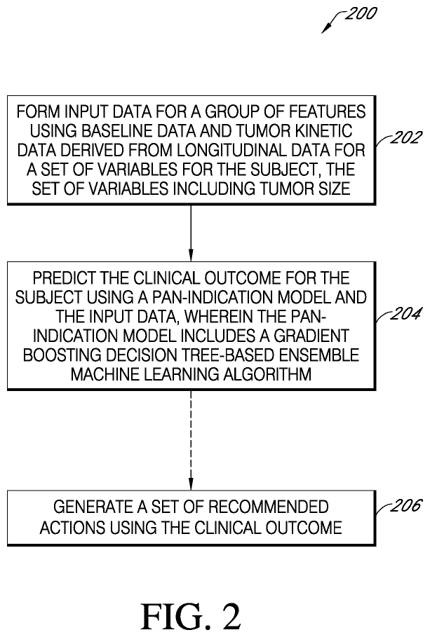

FIG. 2 is a flowchart of a process 200 for predicting a clinical outcome for a subject in accordance with various embodiments. In various embodiments, process 200 is implemented using clinical outcome prediction system 100 described in FIG. 1.

Step 202 includes forming input data for a group of features using baseline data and tumor kinetic data derived from longitudinal data for a set of variables for the subject, the set of variables including tumor size. This input data may be one example of an implementation for input data 116 in FIG. 1. The longitudinal data may be one example of an implementation for longitudinal data 102 in FIG. 1.

The baseline data includes values for a plurality of baseline parameters (or covariates). These covariates may include, for example, age, gender, baseline C-reactive protein (CRP), and baseline albumin, as well as other types of covariates. In one or more examples, the baseline data includes data for 40 or more covariates or baseline parameters.

The longitudinal data for the set of variables corresponds to a period of time, which may also be referred to as an initial period of time. This initial period of time may be, for example, one week, one month, two months, 3 months, 6 months, or some other period of time. In various embodiments, the longitudinal data corresponds to an initial period of time that includes a portion of time after the administration of a treatment, as well as at least one point in time prior to treatment. As one example, the longitudinal data may include a baseline value for a tumor-related variable (e.g., collected pre-treatment) and values for the tumor-related variable for a portion of time (e.g., 4 weeks) post-treatment. The set of variables may include only a single tumor-related variable (e.g., tumor size) or may include multiple tumor-related variables, including tumor size.

The tumor kinetic data derived from the longitudinal data includes values for a plurality of tumor kinetic parameters. These tumor kinetic parameters may include, but are not limited to, tumor growth inhibition (TGI) metrics. In various embodiments, the tumor kinetic data includes values computed or otherwise derived from the longitudinal data for time to tumor regrowth (TTG), tumor growth rate (KG), tumor shrinkage rate (KS), or a combination thereof.

The input data for the group of features is formed in a manner that can be sent to a pan-indication model. In various embodiments, the group of features includes at least three features selected from the group consisting of tumor growth rate (KG), C-reactive protein level (CRP), time to tumor regrowth (TTG), baseline neutrophil/lymphocyte ratio (BNLR), baseline Eastern Cooperative Oncology Group score (ECOG) score, liver metastasis level (LIVER), tumor shrinkage rate (KS), hemoglobin level (HGB), time since initial diagnosis (TSD), total protein (TPRO), albumin level (ALBU), and number of metastatic sites at enrollment (METSITES). The time since initial diagnosis, when in years, may be referred to as years since initial diagnosis (YSD). In some embodiments, the group of features includes all 12 of these features. The time to tumor regrowth, the tumor growth rate, and tumor shrinkage rate, which are tumor kinetic parameters, thus form a subset of the group of features included in the input data.

Step 204 includes predicting the clinical outcome for the subject using a pan-indication model and the input data, wherein the pan-indication model includes a gradient boosting decision tree-based ensemble machine learning algorithm. The pan-indication model may be, for example, pan-indication model 114 in FIG. 1. In various embodiments, the pan-indication model is XGBoost. The pan-indication model receives the input data, processes this input data, and generates a clinical outcome output (e.g., clinical outcome output 118 in FIG. 1). This clinical outcome output may include, for example, one or more hazard ratios. In various embodiments, the clinical outcome output includes a hazard ratio for overall survival.

The pan-indication model is one that has been trained using data for a selected population to process the input data and generate the clinical outcome output. The selected population may include multiple patient/subject populations across at least one clinical trial across at least one of a plurality of tumor types or a plurality of treatment types. In some embodiments, the selected population may include multiple patient/subject populations across a plurality of tumor types, a plurality of treatment types, or both.

As described above, in various embodiments, the input data that is sent into the pan-indication model includes values for 12 features that are listed below in Table 1. These 12 features are listed in Table 1 in decreasing order of impact to the clinical outcome output generated by the pan-indication model, as determined by a Shapely Additive Explanations (SHAP) analysis of the trained pan-indication model. Further, these 12 features do not include a feature for tumor type or treatment protocol (or type). Accordingly, the clinical outcome output generated by the pan-indication model may be considered independent or agnostic of tumor type and treatment protocol (or type).

TABLE 1

| Index | Feature |
|---|---|
| 1 | Tumor growth rate (KG) |
| 2 | C-reactive protein level (CRP) |
| 3 | Time to tumor regrowth (TTG) |
| 4 | Baseline neutrophil/lymphocyte ratio (BNLR) |
| 5 | Baseline Eastern Cooperative Oncology Group score (BECOG) |
| 6 | Liver metastasis level (or liver metastasis status) (LIVER) |
| 7 | Tumor shrinkage rate (KS) |
| 8 | Hemoglobin level (HGB) |
| 9 | Time since initial diagnosis (TSD; when in years: YSD) |
| 10 | Total protein level (TPRO) |
| 11 | Albumin (ALBU) |
| 12 | Number of metastatic sites at enrollment (METSITES) |

In some embodiments, the clinical outcome output generated by the pan-indication model in step 204 provides an indication of clinical outcome (e.g., overall survival). In other embodiments, the clinical outcome output is further processed and then used to make a prediction of the clinical outcome.

In various embodiments, the clinical outcome predicted in step 204 is used to generate one or more hazard ratios of the treatment arm with respect to the control arm. For example, the clinical outcome output generated by the pan-indication model may be a score that indicates overall survival. This score may then be used to generate a hazard ratio for overall survival.

In various embodiments, process 200 optionally includes step 206. Step 206 includes generating a set of recommended actions using the clinical outcome predicted in step 204. The set of recommended actions may be, for example, without limitation, set of recommended actions 122 in FIG. 1. For example, the clinical outcome may be used to determine whether or not a current treatment protocol for the subject should be switched or adjusted. Adjusting a treatment protocol may include, but is not limited to, increasing or decreasing a dosage, increasing or decreasing an interval between doses, or a combination thereof.

As one specific example, step 206 includes determining whether the clinical outcome (and/or clinical outcome output) meets a set of criteria. When the clinical outcome output is a score, the set of criteria may include, for example, without limitation, being below a selected threshold. In this example, the clinical outcome (and/or clinical outcome output) meeting the set of criteria may cause a recommendation that a change from a current treatment protocol to a different treatment protocol be made. In this manner, the clinical outcome predicted by the pan-indication model can be used to provide Personalized Health Care.

FIG. 3 is a flowchart of a process 300 for forming input data for a pan-indication model in accordance with various embodiments. Process 300 describes one example of a manner in which step 202 of process 200 may be performed. In various embodiments, process 300 is implemented using clinical outcome prediction system 100 described in FIG. 1.

Step 302 includes identifying longitudinal data for a set of variables for the subject, the set of variables including tumor size. As previously described with respect to step 202 in FIG. 1, the longitudinal data for the set of variables corresponds to a period of time, which may also be referred to as an initial period of time. This initial period of time may include a baseline portion of time and a post-treatment portion of time. The baseline portion of time may be a single point in time or interval of time prior to the administration of a treatment. The post-treatment portion of time is an interval or period of time that occurs after the administration of a treatment. The initial period of time may be, for example, one week, one month, two months, 3 months, 6 months, or some other period of time. The set of variables includes a set of tumor-related variables, including tumor size.

Step 304 includes modeling a trajectory of the set of variables over a period of time to derive values for a plurality of tumor kinetic parameters. This period of time may also be referred to as a modeled period of time. The modeled period of time may be varied and based on the subject. In various embodiments, the modeled period of time includes the portion of time after the initial period of time and up to a timepoint at which the expected cancer progression for the tumor growth reaches a selected threshold or meets selected criteria. The modeled period of time may be in months or years.

The values derived for the tumor kinetic parameters may be collectively referred to as tumor kinetic data. The tumor kinetic parameters may include, for example, but are not limited to, two or more of a time to tumor regrowth, tumor growth rate, and tumor shrinkage rate.

Step 306 includes forming input data for a group of features using the values for the plurality of tumor kinetic parameters and baseline data. The baseline data includes values for covariates. These covariates may include, for example, without limitation, age, gender, baseline C-reactive protein (CRP), and baseline albumin, as well as other types of covariates. In one or more examples, the baseline data includes data for over 40 covariates.

In one or more embodiments, the input data is comprised of the tumor kinetic data and the baseline data. In other embodiments, the input data is formed by transforming the tumor kinetic data and the baseline data. For example, the tumor kinetic data and the baseline data may be preprocessed and represented in a manner that is compatible with the pan-indication model into which this data is to be input.

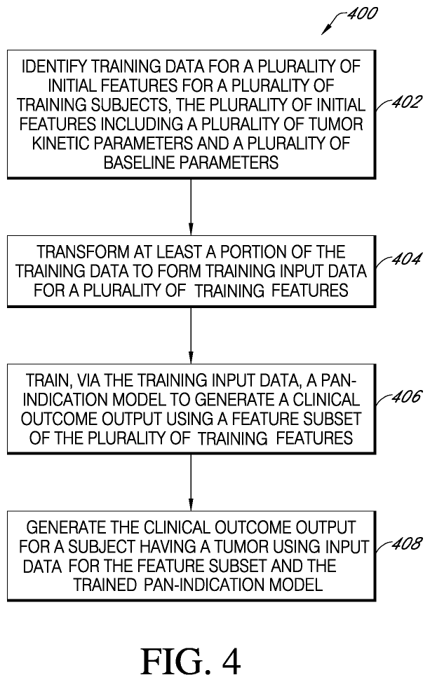

FIG. 4 is a flowchart of a process 400 for training a pan-indication model and using the trained pan-indication model to predict clinical outcomes in accordance with various embodiments. In various embodiments, process 400 is implemented using clinical outcome prediction system 100 described in FIG. 1.

Step 402 includes identifying training data for a plurality of initial features for a plurality of training subjects, the plurality of initial features including a plurality of tumor kinetic parameters and a plurality of baseline parameters. The tumor kinetic parameters include, for example, but are not limited to, two or more of a time to tumor regrowth, tumor growth rate, and tumor shrinkage rate. The baseline parameters may include, for example, C-reactive protein level, baseline neutrophil/lymphocyte ratio, baseline ECOG score, liver metastasis level, tumor shrinkage rate, hemoglobin level, time since initial diagnosis, total protein, albumin level, number of metastatic sites at enrollment, or a combination thereof. In some cases, the baseline parameters include C-reactive protein level, baseline neutrophil/lymphocyte ratio, baseline ECOG score, liver metastasis level, tumor shrinkage rate, hemoglobin level, time since initial diagnosis, total protein, albumin level, number of metastatic sites at enrollment, and one or more other baseline parameters. In some cases, the baseline parameters include 41 baseline parameters.

The training subjects include subjects having different types of tumors (or cancers) (e.g., non-small cell lung cancer (NSCLC), small cell lung cancer (SCLC), renal cell carcinoma (RCC), bladder transitional cell carcinoma (TCC), triple-negative breast cancer (TNBC), etc.). The training subjects may also include subjects that have underwent different types of treatment protocols (e.g., at least two different types of therapeutics). In some embodiments, the training subjects include one or more subjects that have underwent multiple treatment protocols at a same type.

Step 404 includes transforming at least a portion of the training data to form training input data for a plurality of training features. The training input data may be one example of an implementation for input data 116 in FIG. 1 and at least a portion of process 300 described in FIG. 3 may be used to implement step 404. The transformation in step 404 may include, for example, one-hot encoding of parameters that are discrete (not continuous). For example, the transformation may include one-hot encoding of any of the baseline parameters that are categorical variables. With one-hot encoding, a single parameter may be converted into multiple features. Thus, the plurality of training features formed in step 404 is greater in number than the plurality of initial features described in step 402.

In various embodiments, step 404 may include selectively excluding one or more baseline parameters, normalizing values for one or more of the tumor kinetic parameters and/or baseline parameters, processing the training data in some other manner, or a combination thereof. In some examples, the training data identified in step 402 may include data from various sources and/or in one or more different formats. In these examples, step 404 may include combining this data and/or performing one or more preprocessing steps to form the training input data for the plurality of training features. For example, the data for two different initial features may be preprocessed in some manner to form the training input data for a single training feature or for more than two training features.

Step 406 includes training, via the training input data, a pan-indication model to generate a clinical outcome output using a feature subset of the plurality of training features. The pan-indication model may be, for example, pan-indication model 114 in FIG. 1. The pan-indication model may include a gradient boosting model. In various embodiments, this gradient boosting model is an XGBoost model.

Step 406 may be performed in different ways. In one or more examples, the plurality of training features includes over 100 features (e.g., 141 features). Step 406 may include performing an initial training using the plurality of training features. After initial training, a feature subset of the training features having the most impact on the clinical outcome output generated by the pan-indication model is identified. For example, without limitation, the impact of each training feature to the clinical outcome output may be computed. The impact may be computed via SHAP analysis of the pan-indication model based on the initial training. A selected number (e.g., 3, 5, 10, 12, 15, etc.) of the most impactful training features are chosen for addition to the feature subset. The feature subset may include, for example, at least 3 or more features of those listed above in Table 1. As one example, the feature subset may include the 12 features listed above in Table 1.

The pan-indication model is then trained using the feature subset to generate the clinical outcome output. In this manner, the training of the pan-indication model is performed in a multilayered manner.

The feature subset refrains from using features that include or otherwise indicate tumor type or treatment protocol (or type). Accordingly, the clinical outcome output generated by the pan-indication model using the feature subset is considered independent or agnostic of tumor type and treatment protocol (or type).

Step 408 includes generating the clinical outcome output for a subject having a tumor using input data for the feature subset and the trained pan-indication model. This input data may be formed from data that is obtained for the subject, including baseline data and longitudinal data. This input data may be formed in a manner similar to the formation of input data described above with respect to step 202 in FIG. 2 above. The trained pan-indication model may be capable of predicting the clinical outcome for the subject with a high level of accuracy regardless of whether the tumor is a novel or newly identified tumor, regardless of whether a treatment protocol being administered for the subject is a novel or newly identified, or both.

### III. Computer Implemented System

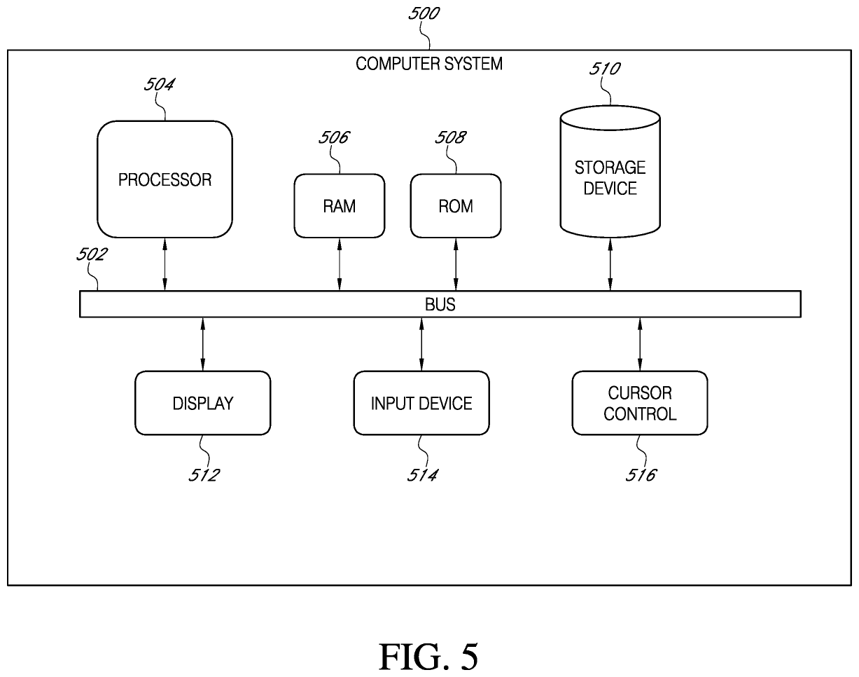

FIG. 5 is a block diagram of a computer system in accordance with various embodiments. Computer system 500 may be an example of one implementation for computing platform 106 described above in FIG. 1. In one or more examples, computer system 500 can include a bus 502 or other communication mechanism for communicating information, and a processor 504 coupled with bus 502 for processing information. In various embodiments, computer system 500 can also include a memory, which can be a random access memory (RAM) 506 or other dynamic storage device, coupled to bus 502 for determining instructions to be executed by processor 504. Memory also can be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A storage device 510, such as a magnetic disk or optical disk, can be provided and coupled to bus 502 for storing information and instructions.

In various embodiments, computer system 500 can be coupled via bus 502 to a display 512, such as a cathode ray tube (CRT) or liquid crystal display (LCD), for displaying information to a computer user. An input device 514, including alphanumeric and other keys, can be coupled to bus 502 for communicating information and command selections to processor 504. Another type of user input device is a cursor control 516, such as a mouse, a joystick, a trackball, a gesture input device, a gaze-based input device, or cursor direction keys for communicating direction information and command selections to processor 504 and for controlling cursor movement on display 512. This input device 514 typically has two degrees of freedom in two axes, a first axis (e.g., x) and a second axis (e.g., y), that allows the device to specify positions in a plane. However, it should be understood that input devices 514 allowing for three-dimensional (e.g., x, y, and z) cursor movement are also contemplated herein.

Consistent with certain implementations of the present teachings, results can be provided by computer system 500 in response to processor 504 executing one or more sequences of one or more instructions contained in RAM 506. Such instructions can be read into RAM 506 from another computer-readable medium or computer-readable storage medium, such as storage device 510. Execution of the sequences of instructions contained in RAM 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 504 for execution. Such a medium can take many forms, including but not limited to, non-volatile media, volatile media, and transmission media. Examples of non-volatile media can include, but are not limited to, optical, solid state, magnetic disks, such as storage device 510. Examples of volatile media can include, but are not limited to, RAM 506 (e.g., dynamic RAM (DRAM) and/or static RAM (SRAM)). Examples of transmission media can include, but are not limited to, coaxial cables, copper wire, and fiber optics, including the wires that comprise bus 502.

Additionally, a computer-readable medium may take various forms such as, for example, but not limited to, a floppy disk, a flexible disk, hard disk, magnetic tape, or any other magnetic medium, a CD-ROM, any other optical medium, punch cards, paper tape, any other physical medium with patterns of holes, RAM, PROM, EPROM, EEPROM, FLASH-EPROM, solid-state memory, one or more storage arrays (e.g., flash arrays connected over a storage area network), network attached storage, any other memory chip or cartridge, or any other tangible medium from which a computer can read.

In addition to computer readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 500 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 500, whereby processor 504 would execute the analyses and determinations provided by these engines, subject to instructions provided by any one of, or a combination of, the memory components RAM 506, ROM, 508, or storage device 510 and user input provided via input device 514.

### IV. Examples and Results

IVA. Methodology

Longitudinal data for tumor size and baseline data was compiled from 10 cancer clinical trials of atezolizumab (N=8121 patients). This data formed a multi-cancer dataset. The longitudinal data was used to derive the TGI metrics of time to tumor regrowth, tumor growth rate, and tumor shrinkage rate. The baseline data included data for 41 baseline variables. Any categorical variables were on-hot encoded to thereby produce input data for a final set of 141 features. Both linear and nonlinear machine learning models were used to process the input data and predict overall survival. These models included a Cox Proportional Hazards (CoxPH) model, CoxNet model (a regularized Cox regression model), random forest model, gradient boosting model, and an XGBoost model (e.g., pan-indication model 114 in FIG. 1). For non-XGBoost models, mean imputation was used for missing data. The data was split into training and testing sets. Parameter tuning was performed via 5-fold cross validation on the training set.

Harrell's Concordance (C)-index was used for evaluation. The top 12 features of the full set of 141 features were selected for the final XGBoost model.

IVB. Results

Using TGI metrics to predict overall survival improved the predictive performance of all models. When trained on the final set of 141 features, the XGBoost model outperformed all other models when TGI metrics were included in the analysis (C-index of 0.81). The XGBoost model retained a high C-index of 0.80 even after the number of input features was restricted to the top 12 most impactful features. These 12 features did not include treatment protocol or cancer type.

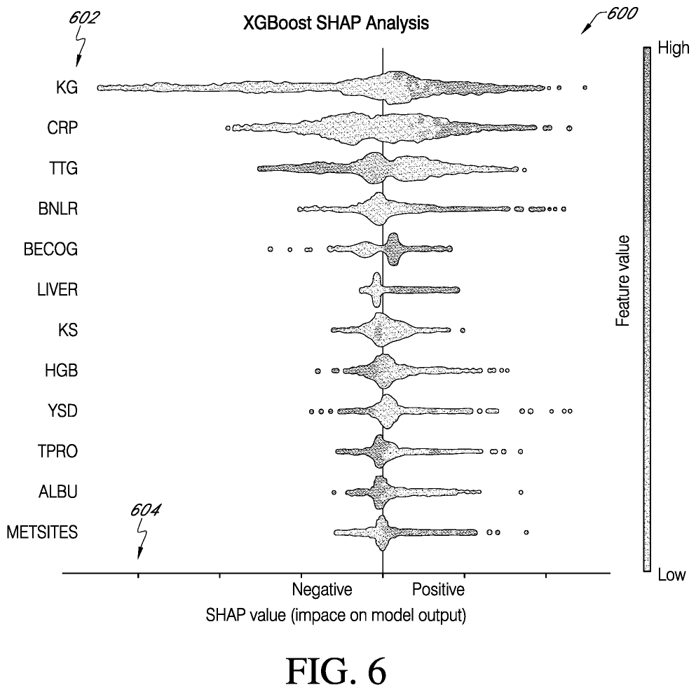

FIG. 6 is a plot 600 illustrating the relative importance of features used as input for the XGBoost model in accordance with various embodiments. Plot 600 illustrates the relative impact of 12 features on the clinical outcome output generated by the XGBoost model trained in the manner described above. Plot 600 includes y-axis 602 identifying features and x-axis 604 identifying each feature's corresponding SHapley Additive exPlanations (SHAP) value. The SHAP value indicates the respective feature's impact on the clinical outcome output generated by the pan-indication model. The higher the SHAP value, the greater the impact that the feature had on the clinical outcome output. These 12 features, also identified in Table 1 above, were used to generate the final XGBoost model.

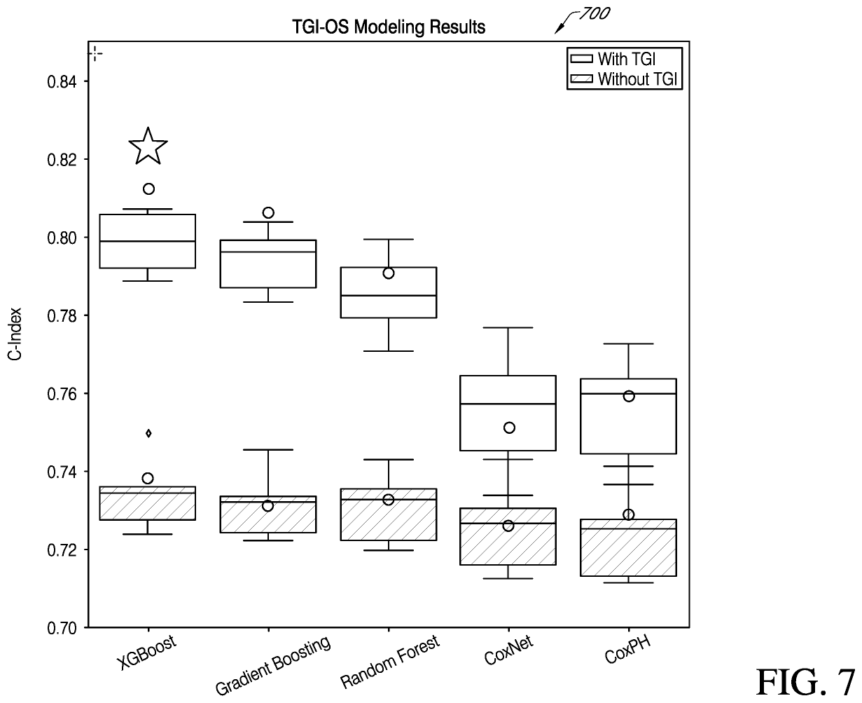

FIG. 7 is a plot 700 illustrating the accuracy of the XGBoost model as compared to the other models in accordance with various embodiments. Plot 700 illustrates that the XGBoost model outperformed the regular gradient boosting model, the random forest model, the CoxNet model, and the Cox Proportional Hazards (CoxPH) model. Specifically, the XGBoost outperformed these other models especially when the TGI metrics were used.

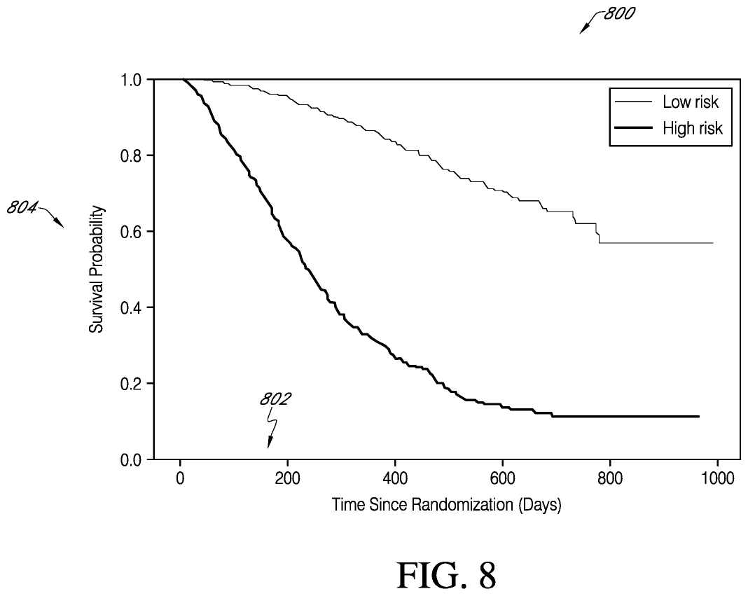

FIG. 8 is a plot 800 illustrating the XGBoost's ability to stratify patients based on individual predictions in accordance with various embodiments. Plot 800 includes x-axis 802 that identifies time since randomization in days and y-axis 804 that identifies survival probability. As shown, subjects with a low predicted hazard rate (e.g., low risk in the lower 50^{th} percentile) were easily distinguishable from subjects with a high predicated hazard rate (e.g., high risk in the upper 50^{th} percentile).

### V. Exemplary Descriptions of Terms

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. Section divisions (e.g., heading and/or subheadings) in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements.

As used herein, “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. “Subject” and “patient” are used interchangeably herein with respect to various embodiments.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, a “model” may include one or more algorithms, one or more mathematical techniques, one or more machine learning algorithms, or a combination thereof.

As used herein, “machine learning” may be the practice of using algorithms to parse data, learn from it, and then make one or more determinations or predictions about something in the world. Machine learning uses algorithms that can learn from data without relying on rules-based programming. A machine learning model may be a model that uses such algorithms to process input data and generate an output. Machine learning may be part of artificial intelligence.

As used herein, a “pan-indication model” may be a machine learning model capable of providing a prediction or indication of a clinical outcome independent of tumor type. In various embodiments, the pan-indication model is also capable of providing this indication independent of treatment type. The pan-indication model may include a gradient boosting model and therefore may also be referred to as a pan-indication gradient boosting model. In various embodiments, the pan-indication model includes a gradient boosting decision tree-based ensemble machine learning algorithm. In various embodiments, the pan-indication model is implemented using Extreme Gradient Boosting (XGBoost) and may be referred to as a pan-indication XGBoost model.

As used herein, a “clinical outcome’ is a measurable change in health, function, or quality of life that results from an action. This action may be, for example, a one-time event, a periodic event, or an ongoing event. For example, the action may be a chemotherapy treatment, a therapeutic or drug treatment, or some other type of treatment or treatment protocol.

As used herein, a “treatment protocol” may describe the strategy associated with a particular type of treatment. Examples of treatments include, but are not limited to, surgery, chemotherapy, radiation therapy, stem cell or bone marrow transplantation, immunotherapy, hormone therapy, and targeted drug (e.g., therapeutic) therapy. A treatment protocol may include, for example, the plan or methodology associated with administering the particular type of treatment to a subject. For example, this plan or methodology may include a schedule for the administration of the treatment (e.g., an interval between doses), a dosing or administration amount, and/or other relevant factors.

As used herein, “longitudinal data” may include data over or corresponding to a period of time. The period of time may be in days, weeks, months, years, or some other measure of time.

As used herein, “baseline data” may include data collected at a point in time or over a period of time prior to treatment (or pre-treatment).

As used herein, a “covariate” may be a predictor or explanatory variable. For example, a covariate may be a feature associated with a subject that is used to predict one or more outcomes. In the machine learning context, a set of covariates may be a set of independent variables (or features) that can help to explain or predict the outcome/dependent variable.

As used herein, “overall survival” with respect to a particular cause (e.g., tumor growth) may mean that the subject remains alive and does not die from that cause. When data for overall survival is being used to train a machine learning model, the data may include censored data in which the recorded overall survival for a subject is the time the subject is known to have survived. For example, when definitive data regarding the length of time a subject was or is alive post-diagnosis or post-treatment is unattainable, the date for that subject's last follow-up or another data at which the subject was known to be alive may be used.

As used herein, a “hazard ratio” (or Hazard Ratio or HR) may be a measure of how often a particular event happens in one group (e.g., a treatment group) compared to how often it happens in another group (e.g., a control group), over time. For example, in tumor or cancer research, hazard ratios may be used in clinical trials to measure survival at any point in time in a group of patients who have been given a specific treatment compared to a control group given another treatment or a placebo. A hazard ratio can be used to provide an indication of overall survival. For example, a hazard ratio of one means that there is no difference in survival between the two groups. A hazard ratio of greater than one or less than one means that survival was better in one of the groups.

As used herein, a “score” may include a number, a probability, a metric, indicator, plot, graphic, notification, output, another type of output, or a combination thereof.

As used herein, “form”, “forms” or “forming” may refer to creating, generating, processing, analyzing, modifying, extracting, accessing, identifying, providing, producing, constructing, or a combination thereof.

### VI. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications, alternatives, and equivalents are possible within the scope of the invention claimed. Thus, it should be understood that although the present invention as claimed has been specifically disclosed by embodiments and optional features, modifications, variations, and/or equivalents of the concepts herein disclosed may be resorted to by those skilled in the art, and that such modifications, variations, and/or equivalents are considered to be within the scope of this invention as defined by the appended claims.

The description provides preferred exemplary embodiments only, and is not intended to limit the scope, applicability, or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims. For example, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Specific details may be provided to provide a thorough understanding of the embodiments. However, it will be understood that the embodiments may be practiced without these specific details. For example, circuits, systems, networks, processes, or other components may be shown as components in block diagram form in order not to obscure the embodiments in unnecessary detail. In other instances, well-known circuits, processes, algorithms, structures, or techniques may be shown without unnecessary detail in order to avoid obscuring the embodiments.

What is claimed is:

1. A method for predicting a clinical outcome for a subject, the method comprising:
identifying longitudinal data for a set of variables for the subject, the set of variables including tumor size;
modeling a trajectory of the set of variables over a period of time using the longitudinal data to derive values for a plurality of tumor kinetic parameters;
forming input data for a group of features using the values for the plurality of tumor kinetic parameters and baseline data obtained for the subject prior to treatment; and
predicting the clinical outcome for the subject using a pan-indication gradient boosting model and the input data.

2. The method of claim 1, wherein the clinical outcome comprises overall survival and wherein the plurality of tumor kinetic parameters includes at least two parameters selected from a group consisting of a time to tumor regrowth, a tumor growth rate, and a tumor shrinkage rate.

3. The method of claim 2, wherein the group of features is agnostic with respect to tumor type and wherein the pan-indication gradient boosting model generates a clinical outcome output that provides an indication of the overall survival independent of the tumor type.

4. The method of claim 2, wherein the group of features is agnostic with respect to treatment protocol and wherein the pan-indication gradient boosting model generates a clinical outcome output that provides an indication of the overall survival independent of the treatment protocol.

5. The method of claim 1, further comprising:
training the pan-indication gradient boosting model using data for a plurality of training features for a plurality of training subjects, the data being collected from a plurality of clinical trials across at least one of a plurality of tumor types or a plurality of treatment types.

6. The method of claim 1, wherein the baseline data comprises values for a plurality of baseline parameters and further comprising:
identifying training data for a plurality of initial features for a plurality of training subjects, the plurality of initial features including the plurality of tumor kinetic parameters and the plurality of baseline parameters, wherein the training data is collected from a plurality of clinical trials across at least one of a plurality of tumor types or a plurality of treatment types; and
transforming at least a portion of the training data to form training input data for a plurality of training features for use in training the pan-indication gradient boosting model.

7. The method of claim 6, further comprising:
training the pan-indication gradient boosting model using the training input data using a feature subset of the plurality of training features, wherein the group of features is included in the feature subset.

8. The method of claim 1, further comprising:
generating a set of hazard ratios for the subject using a clinical outcome output generated by the pan-indication gradient boosting model.

9. The method of claim 1, wherein predicting the clinical outcome comprises:
generating a clinical outcome output using the pan-indication gradient boosting model and the input data; and
generating a set of hazard ratios for the subject using the clinical outcome output.

10. The method of claim 1, further comprising:
determining whether a clinical outcome output generated by the pan-indication gradient boosting model meets a set of criteria; and
switching from a current treatment protocol for the subject to a different treatment protocol in response to a determination that the clinical outcome output meets the set of criteria.

11. The method of claim 1, further comprising:
generating a set of recommended actions based on the clinical outcome predicted by the pan-indication gradient boosting model.

12. The method of claim 1, wherein the group of features includes at least three features selected from the group consisting of tumor growth rate, C-reactive protein level, time to tumor regrowth, baseline neutrophil/lymphocyte ratio, baseline ECOG score, liver metastasis level, tumor shrinkage rate, hemoglobin level, time since initial diagnosis, total protein, albumin level, number of metastatic sites at enrollment.

13. A method for predicting a clinical outcome for a subject, the method comprising:
forming input data for a group of features using baseline data and tumor kinetic data derived from longitudinal data for a set of variables for the subject, the set of variables including tumor size; and
generating a clinical outcome output that provides an indication of the clinical outcome for the subject using a pan-indication model and the input data, wherein the pan-indication model includes a gradient boosting decision tree-based ensemble machine learning algorithm.

14. The method of claim 8, further comprising:
obtaining the baseline data and the longitudinal data for the subject, wherein the longitudinal data corresponds to a period of time that includes a portion of time after administration of a treatment.

15. The method of claim 8, wherein the pan-indication gradient boosting model includes Extreme Gradient Boosting (XGBoost).

16. A method for generating a clinical outcome output for a subject having a tumor, the method comprising:
forming input data for a group of features using baseline data and tumor kinetic data derived from longitudinal data for a set of variables for the subject, the set of variables including tumor size,
wherein the group of features includes at least three features selected from features comprising:
tumor growth rate (KG),
C-reactive protein level (CRP),
time to tumor regrowth (TTG),
baseline neutrophil/lymphocyte ratio (BNLR),
baseline Eastern Cooperative Oncology Group score (BECOG),
liver metastasis level (LIVER),
tumor shrinkage rate (KS),
hemoglobin level (HGB),
time since initial diagnosis (TSD),
total protein level (TPRO),
albumin (ALBU), and
number of metastatic sites at enrollment (METSITES),
wherein the features are listed in order of decreasing impact on the clinical outcome output; and
generating the clinical outcome output for the subject using a pan-indication model and the input data, wherein the pan-indication model includes a gradient boosting decision tree-based ensemble machine learning algorithm.

17. The method of claim 16, wherein the clinical outcome output provides an indication of overall survival independent of tumor type and treatment protocol.

18. The method of claim 16, wherein the forming comprises:
modeling a trajectory of the set of variables over a period of time using the longitudinal data to derive the tumor kinetic data, wherein the tumor kinetic data includes tumor growth rate and time to tumor regrowth.

19. A method for predicting clinical outcomes for subjects having tumors, the method comprising:
identifying training data for a plurality of initial features for a plurality of training subjects, the plurality of initial features including a plurality of tumor kinetic parameters and a plurality of baseline parameters;
transforming at least a portion of the training data to form training input data for a plurality of training features;
training, via the training input data, a pan-indication gradient boosting model to generate a clinical outcome output using a feature subset of the plurality of training features; and
generating the clinical outcome output for a subject having a tumor using the pan-indication gradient boosting model that has been trained and input data for the feature subset.

20. The method of claim 19, wherein the clinical outcome output provides an indication of overall survival and further comprising at least one of:
generating a hazard ratio for the overall survival using the clinical outcome output; or
generating a set of recommended actions regarding a current treatment protocol for the subject based on the clinical outcome output.
